(19) United States
(12) Patent Application Publication
(10) Pub. No.: US 2004/0248131 A1
(43) Pub. Date: Dec. 9, 2004

(54) Methods for dna mutagenesis and dna cloning

(76) Inventors: Thomas Rudel, Berlin (DE); Nikolaus Machuy, Berlin (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/491,620

(22) PCT Filed: Oct. 2, 2002
(86) PCT No.: PCT/EP02/11086

(30) Foreign Application Priority Data
Oct. 2, 2001 (DE) 101 48 607.3

(51) Int. Cl. C12Q 1/68

(52) U.S. Cl. 435/6

(57) ABSTRACT

The invention relates to methods and reagent kits for DNA manipulation, in particular for site-specific mutagenesis or for cloning.

The present invention relates to methods and reagent kits for DNA manipulation, in particular for site-specific mutagenesis or for cloning.

Despite the considerable progress that has been made in recent years in the manipulation of nucleic acids, for example the mutagenization or cloning of DNA fragments, there is still a considerable need for improving methods of this kind. Known mutagenization and cloning methods often have problems with respect to their efficiency. The present application provides novel mutagenization and cloning methods which can at least partially eliminate these problems.

In order to study structure-function relationships such as, for example, kinase action as a function of particular amino acids or the promoter action of DNA sections as a function of their sequence, it is often necessary to carry out specific mutagenesis of nucleic acids.

Hutchison et al. (Hutchison et al., 1978) describe a site-directed mutagenesis in which single-stranded DNA is hybridized with an oligonucleotide which is complementary to the template DNA, with the exception of the base to be mutagenized. After extension of the oligonucleotide, i.e. generation of a double strand by using a DNA polymerase, closing of the synthesized strand by using a ligase and transformation into E. coli, 50% of the clones really should contain mutated DNA. In practice, however, this rate is distinctly lower (Kramer et al., 1984).

Therefore, the currently commercially available mutagenesis systems use selection marker for the mutated DNA. Some of these are to be introduced here. The pALTER® system from Promega uses a vector which comprises in its initial state an inactive ampicillin resistance gene and an active tetracycline resistance gene. The mutagenesis is carried out in a manner similar to the above-described method, with the difference that, together with the mutagenesis oligonucleotide, two further primers are hybridized which activate the Amp^{R} gene and inactivate the Tet^{R} gene (Kleina et al., 1990; Normanly et al., 1990). After transformation into E. coli, the clones containing the mutated DNA are selected using ampicillin. Disadvantages of this method are, on the one hand, that the DNA to be mutated must be cloned into a special plasmid and, on the other hand, that DNA repair-deficient E. coli strains must be used.

The Transformer™ site-directed mutagenesis kit from Clontech uses for selection a second oligonucleotide to be hybridized, which is used to destroy a unique restriction cleavage site in the starting construct (Deng and Nickoloff, 1992; Zhu, 1996). After hybridization and oligonucleotide extension, digestion with this restriction enzyme cuts only the nonmutated plasmids which are thus no longer transformable. This method also makes use of repair-deficient strains. Moreover, two sequential transformations are required, and this substantially prolongs the mutagenesis.

The mutagenesis using the Quickchange™ site-directed mutagenesis kit from Stratagene involves hybridizing two complementary mutagenesis oligonucleotides to both strands and amplifying the mutated DNA with the aid of Pfu-Turbo DNA polymerase in 12 to 18 temperature cycles. After digesting the template DNA with DpnI, the double-stranded DNA is transformed into E. coli which then closes the single-strand gaps still present.

In a further mutagenesis method, the DNA section to be mutated is amplified in two separate PCRs in such a way that the products overlap by approx. 20 bases at the site of mutation and already contain the mutated sequence (Lottspeich, F. and Zorbas, 1998). The two products are mixed and the entire DNA is amplified in a third PCR using oligonucleotides which allow cloning into the desired vector. The disadvantage of this method is the fact that three PCRs are required.

Another mutagenesis method (Cooney, 1998) likewise starts with two PCRs. In this method too, the two products overlap at the site of mutation, albeit not by 20 bases but only by as many for a T₄ DNA polymerase trimming reaction to produce complementary protruding 5′ ends. The outer ends of the PCR product are treated with restriction enzymes. The two products and the vector are mixed for ligation. Disadvantages of this method are both the fact that restriction cleavage sites within the fragments must be taken into consideration and the ligation of three DNA fragments.

A first aspect of the present application is the subject matter of claim 1 and relates to a method for mutagenizing nucleic acids, in particular double-stranded DNA fragments, which comprises the following steps:

(a) providing a circular double-stranded nucleic acid template to be mutagenized, for example a plasmid vector,

(b) providing two oligonucleotides which

(i) hybridize in each case with one strand of said nucleic acid template and

(ii) have in the region of their 5′ ends a short section, preferably of 2-4 base pairs, complementary to one another,

(iii) with at least one of said oligonucleotides comprising the mutated target sequence which is preferably located in the region of the 5′ end of said oligonucleotide,

(c) amplifying the nucleic acid template to be mutagenized of (a) by using the oligonucleotides having the mutated target sequence of (b) as primers, obtaining a mutated double-stranded amplification product,

(d) trimming the ends of said amplification product, generating a trimmed amplification product having protruding 5′ ends complementary to one another, and

(e) ligating said trimmed amplification product, obtaining a mutated circular nucleic acid construct.

Preferred embodiments of this method are subject matter of claims 2 to 6.

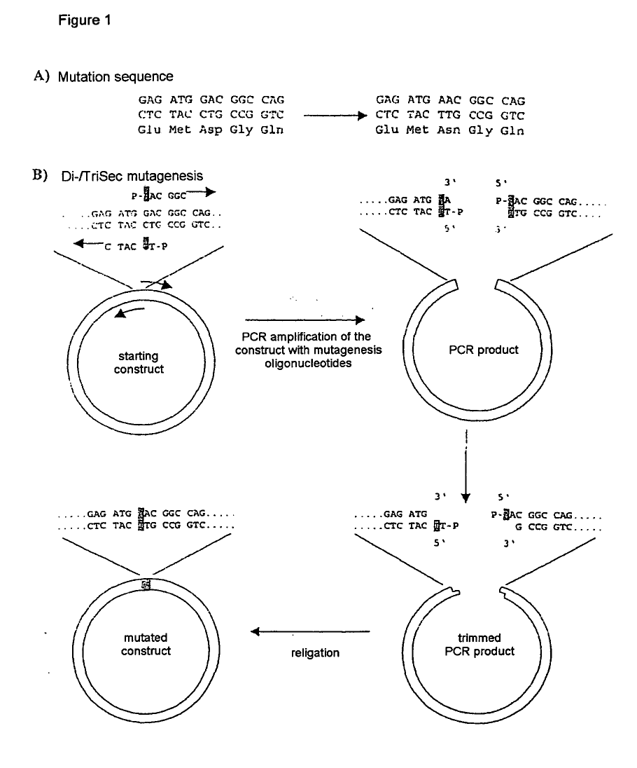

With the aid of said method, a novel, very simple, inexpensive and efficient mutagenesis method is provided (FIG. 1). To this end, oligonucleotides are prepared which comprise, for example at the 5′ ends, the mutated DNA sequences and which have preferably a 2-3 base pair overlap (in this case AA and TT, respectively). The length of the oligonucleotides is chosen so as for the latter to be able to bind with adequate efficiency to the template under suitable hybridization conditions. The length of the oligonucleotides is usually 15-50 bases, for example. PCR amplification of the entire construct, using the mutagenesis oligonucleotides, and subsequent trimming of the ends, for example by T₄ DNA polymerase using the appropriate stop nucleotide (dGTP in this example), produce a product having protruding 5′ ends complementary to one another which can be ligated very efficiently.

The rate of mutagenesis can be further increased by removing the template DNA, for example by means of digestion with DpnI which cuts only methylated DNA. A negative control which may be used is a ligation mixture without ligase. After transformation into E. coli, the efficiency of mutagenesis can be estimated from the ratio of the number of clones. Ideally, the mixture without ligase is unable to produce any transformants. After transformation of the mutagenized construct into a suitable host cell, the mutation can be checked, for example by sequencing or/and by PCR control, the latter method using an oligonucleotide which produces a product only with mutated DNA. For the first ten mutageneses carried out using this method, the rate of mutation was 95%, with two clones of each mutagenesis having been checked.

The mutagenesis method of the invention is used in particular for carrying out DNA manipulations selected from the group consisting of point mutations, involving an exchange or substitution of one or more single nucleotides; insertions, involving additional introduction of one or more single nucleotides into the sequence; inversions, involving inversion of one or more sequences of at least two bases, for example of two, three or four bases; deletions, involving the removal of one or more single bases; and random combinations of said possible mutageneses, for example double or multiple combinations of point mutation, insertion, inversion or/and deletion in a single step or introducing a plurality of point mutations at maximum distance, corresponding to the length of the oligonucleotides used, of 50 bases, for example, in a single step.

The advantage of the method of the invention over other mutagenesis methods is the extremely high rate of mutagenesis of 95%. Generating staggered ends by trimming, for example by means of a T₄ DNA-polymerase reaction, enables only accurately trimmed products to be religated. In contrast, religation of untrimmed amplification products may be more likely to produce frame shifts due to an additional base, for example an adenine. In contrast to many commercially available systems, the method of the invention does not rely on the presence of unique restriction cleavage sites and of resistances to particular antibiotics. Another advantage of the said method is speed. PCR, T₄ DNA-polymerase reaction, ligation, DpnI digestion and transformation can be carried out on the first day. On the second day, the colonies are analyzed by PCR and cultures for plasmid preparation are prepared. On the third day, the mutated DNA can already be isolated and used in experiments.

Said first aspect of the invention furthermore, as claimed in claim 7, also relates to a reagent kit, in particular for carrying out the above-described mutagenesis method, which kit comprises:

(a) means for nucleic acid amplification,

(b) means for trimming double-stranded nucleic acids to generate staggered ends complementary to one another,

(c) means for ligating nucleic acids and,

(d) where appropriate, means for selectively removing methylated DNA.

The components of the reagent kit may be purchased together or may be assembled from various sources. Furthermore, said kit may comprise customary buffer and auxiliary reagents for carrying out the reactions and a written description of the procedure.

A second aspect of the invention relates to a novel cloning method which may be used to remove in particular the difficulties associated with the formation of multimers when cloning double-stranded nucleic acid fragments, for example oligonucleotides of preferably up to 200 bp, particularly preferably of 15-100 bp, in length or else longer PCR fragments.

The cloning of relatively short DNA fragments normally involves using two complementary oligonucleotides which, after hybridization, have protruding ends complementary to a vector opened with appropriate restriction enzymes and which can be ligated into said vector. However, this frequently incurs the problem of the double-stranded oligonucleotides forming multimers and therefore either being unable to be cloned at all or integrating into the vector as multimers. The method of the invention can circumvent this problem.

Two embodiments are available for generating an opened vector to introduce a double-stranded oligonucleotide or a relatively long DNA fragment:

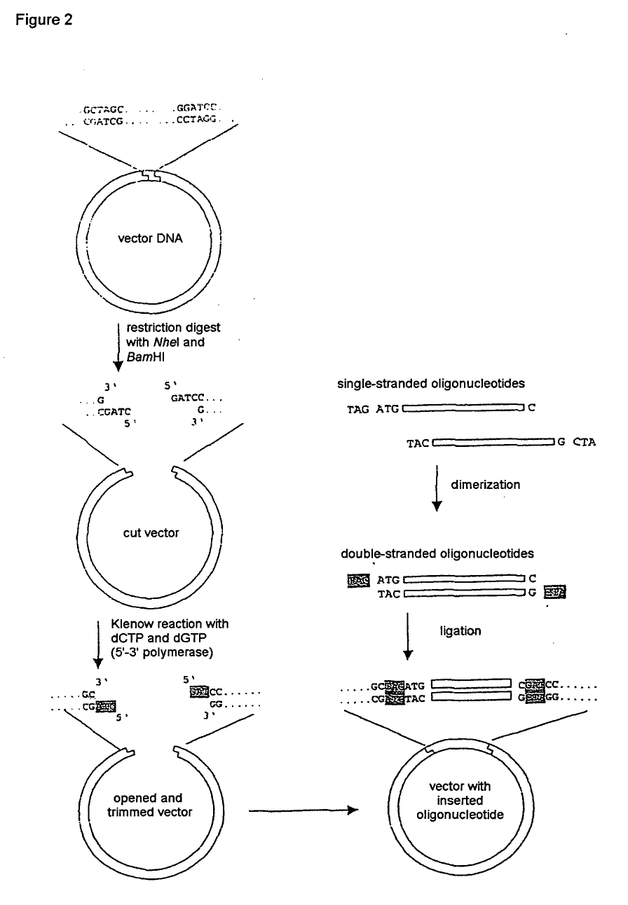

In variant 1, the vector is opened with two different restriction enzymes and one or two nucleotides are filled into the DNA ends with the aid of Klenow polymerase. This produces different staggered ends which are neither complementary to one another nor self-complementary (FIG. 2). The ends of the oligonucleotides are selected so as to be complementary to the filled-in vector ends.

To generate the vector according to variant 2, the part into which the fragment to be cloned is to be inserted is amplified, for example by means of PCR. This is followed by generating staggered ends which are neither self-complementary nor complementary to one another by trimming, for example with T₄ DNA polymerase, using particular stop nucleotides. In order to achieve this, the oligonucleotides must be designed to have suitable bases at the 5′ ends. Variant 2 allows the introduction of double-stranded oligonucleotides into any site in a vector, for example a plasmid.

Double-stranded oligonucleotides are generated by hybridization of in each case equivalent amounts of the single-stranded oligonucleotides to one another under suitable conditions, for example mixing in the presence of MgCl₂, heating and slow cooling from 95° C. to room temperature. The ends of the hybridized oligonucleotides preferably have protruding 5′ ends which are complementary to the protruding 5′ ends of the prepared vectors. Since the protruding 5′ ends of the double-stranded DNA produced in this way are neither complementary to one another nor self-complementary, multimers cannot form. This results in an enormous increase in efficiency of the cloning of oligonucleotides. Oligonucleotide incorporation can be checked by way of PCR or/and restriction digestion and also sequencing.

Another aspect of the invention is subject matter of claim 8 and relates to a method for cloning nucleic acid fragments into a vector according to variant 1, which method comprises the following steps:

(a) providing a circular double-stranded nucleic acid vector,

(b) cleaving the vector of (a) with two different restriction endonucleases, obtaining a linear vector having two different staggered ends,

(c) partially filling in the ends of the cut vector, generating a filled-in linear vector having different staggered ends which are neither complementary to one another nor self-complementary,

(d) providing a nucleic acid fragment to be cloned into the vector of (c), which has two different staggered ends which are complementary to the ends of the filled-in vector but which are neither complementary to one another nor self-complementary, and

(e) ligating said nucleic acid fragment into said filled-in vector.

Preferred embodiments of this method are subject matter of claims 9 to 11.

This embodiment furthermore, as claimed in claim 12, relates to a reagent kit, in particular for carrying out the cloning method, which kit comprises:

(a) means for cleaving a nucleic acid by two different restriction endonucleases which in each case generate different staggered ends,

(b) means for partially filling in staggered ends in double-stranded nucleic acids, for example a suitable enzyme and two nucleoside triphosphates, with the ends being neither complementary to one another nor self-complementary, and

(c) means for ligating nucleic acids.

The reagent kit may furthermore comprise customary buffer and auxiliary reagents and also a written description of the procedure.

Yet another aspect of the invention is subject matter of claim 13 and relates to a method for cloning nucleic acid fragments into a vector according to variant 2, which methods comprises the following steps:

(a) providing a circular double-stranded nucleic acid vector,

(b) providing two oligonucleotides which

(i) in each case hybridize with one strand of the nucleic acid vector and

(ii) have in the region of their 5′ ends a preferably short section, for example of 2-4 base pairs, which is homologous to the ends of the DNA to be introduced,

(c) amplifying the nucleic acid vector of (a) by using the oligonucleotides of (b) as primers, obtaining a double-stranded amplification product,

(d) trimming the ends of said amplification product, generating a trimmed linear vector having different staggered ends which are neither complementary to one another nor self-complementary,

(e) providing a nucleic acid fragment to be cloned into said vector, which has two different staggered ends which are complementary to the ends of the trimmed vector but are neither complementary to one another nor self-complementary, and

(f) ligating said nucleic acid fragment into the said trimmed vector.

Preferred embodiments of this method are subject matter of claims 14 to 15.

This embodiment furthermore, as claimed in claim 16, relates to a reagent kit, in particular for carrying out the abovementioned cloning method, which kit comprises

(a) means for nucleic acid amplification,

(b) means for trimming double-stranded nucleic acids to generate staggered ends which are neither complementary to one another nor self-complementary, and

(c) means for ligating nucleic acids.

The reagent kit may furthermore comprise customary buffer and auxiliary reagents and also a description of the procedure.

Another new possibility in connection with open vectors or plasmid parts generated according to variant 2 comprises introducing amplification products, for example PCR products, trimmed with T₄ DNA polymerase. Moreover, cloning by this method is independent of restriction cleavage sites. The staggered ends of the PCR product to be introduced must be complementary to the vector ends. This makes it possible to introduce particular DNA sections very easily and very precisely and without additional, sometimes interfering bases. This is particularly useful when the fusion proteins are composed of, for example, a signal peptide and the protein to be studied. It is further possible in this way to arbitrarily exchange functional domains between various proteins.

Yet another aspect of the invention is subject matter of claim 17 and relates to a method for cloning nucleic acid fragments into a vector, which comprises the following steps:

(a) providing a circular double-stranded nucleic acid vector having a restriction cleavage site for a restriction enzyme, at which site the recognition sequence is located beside the actual cleavage site,

(b) cleaving the vector with said restriction enzyme,

(c) partially filling in or trimming the ends of the cut vector, generating a linear vector having differently staggered ends which are neither complementary to one another nor self-complementary,

(d) providing a nucleic acid fragment to be cloned into the vector of (c), which has two different staggered ends which are complementary to the ends of the filled-in vector but which are neither complementary to one another nor self-complementary, and

(e) ligating said nucleic acid fragment into said filled-in vector.

This embodiment furthermore, as claimed in claim 18, relates to a reagent kit, in particular for carrying out the abovementioned cloning method, which kit comprises:

(a) means for cleaving a nucleic acid by a restriction endonuclease having a restriction cleavage site at which the recognition sequence is located beside the actual cleavage site,

(b) means for partial filling-in of staggered ends in double-stranded nucleic acids for generating staggered ends which are neither complementary to one another nor self-complementary,

(c) means for trimming double-stranded nucleic acids to generate staggered ends which are neither complementary to one another nor self-complementary, and

(d) means for ligating nucleic acids.

The reagent kit may furthermore comprise customary buffer and auxiliary reagents and also a description of the procedure.

To define the oligonucleotides to be used for mutagenization or cloning, the following rules and algorithms apply with preference:

The properties “not self-complementary” and “not complementary to one another” can be generated by way of choosing suitable sequences at the staggered end.

“Not complementary” to one another means that two staggered ends cannot be ligated with one another.

“Not self-complementary” means that the sequence within a protruding end is not complementary into the staggered end thereof.

When trimming using an enzyme with exonuclease/polymerase activity in the presence of stop nucleotides, for example T₄ DNA polymerase, the overlapping ends must not contain all 4 bases, since otherwise it will be no longer possible to stop the reaction by adding a stop nucleotide.

The preferred annealing temperature of the oligonucleotides is predefined and determined according to the 4+2 rule (2° C. for each adenine and thymine nucleotide; 4° C. for each guanine and cytosine nucleotide) on the basis of the base sequence of the oligonucleotide. The difference in the annealing temperature of the two oligonucleotides should preferably be no more than 2° C.

The base thymidine should preferably be avoided at the 3′ end of the oligonucleotides.

To define oligonucleotides for mutagenesis, the following rules and algorithms are determined:

Trimming, for example by way of a T₄ DNA-polymerase reaction, should produce at the 5′ end of the oligonucleotides protruding ends which are complementary to one another but not self-complementary. The 5′ ends of the amplification products should be phosphorylated.

Either the desired mutation, for example an amino acid substitution, is predefined and the bases to be altered in the process are determined by the algorithm, or only the desired mutation, for example a base substitution, is predefined. In both cases, additional silent mutations may be introduced.

To introduce oligonucleotides at particular sites in a vector, for example a plasmid, a distinction is made between the two possibilities of vector preparation. The following algorithm applies to variant 1, preparation of the vector by restriction digestion and subsequent filling-in reaction, for example using Klenow enzyme:

Possible restriction cleavage sites are predefined. The program is intended to determine a cleavage site combination with predefined nucleotides for the Klenow reaction. In addition, the protruding 5′ ends of the double-stranded oligonucleotides to be introduced, which must be complementary to the vector ends but must not be self-complementary, are to be determined.

If the vector is amplified by PCR according to variant 2, the following rules apply:

The exact site of insertion is predefined. Said site may also comprise a deletion. After T₄ DNA-polymerase reaction, the ends of the PCR products must be neither complementary to one another nor self-complementary. The oligonucleotides used for the PCR must be phosphorylated on their 5′ ends.

If it is not possible to generate at the predefined site of insertion staggered ends having the properties “not self-complementary” and “not complementary to one another”, then, starting from the predefined site of insertion, a sequence should be searched for which allows generation of the desired properties both in the vector and at the ends of the oligonucleotides to be introduced. The ends of the oligonucleotides for the PCR or the introduction are correspondingly extended or truncated. It is also possible to introduce the bases required for cloning via the oligonucleotide.

The length of the oligonucleotides to be introduced is arbitrary and limited only by the given possibilities of oligonucleotide synthesis.

The staggered ends, resulting from dimerization, of the double-stranded oligonucleotides to be introduced must be complementary to the vector ends. In contrast, they must not be self-complementary.

The present invention is furthermore intended to be illustrated by the following examples and figures in which:

FIG. 1 is an example of carrying out the mutagenesis method of the invention. FIG. 1A depicts the mutagenesis to be carried out (replacing G/C with A/T, associated with an amino acid substitution of Asn for Asp). FIG. 1B is a diagrammatic representation of the carrying-out of the method. The starting construct to be mutagenized is amplified, for example by way of PCR. The 5′ ends of the oligonucleotides used as primers for this purpose contain the mutated sequence. A T₄ DNA-polymerase trimming reaction generates staggered complementary ends capable of being ligated with one another. After transformation into a suitable host organism, for example E. coli, the positive clones may be identified using suitable measures such as PCR or/and sequencing.

FIG. 2 depicts the cloning of oligonucleotides according to variant 1 of the cloning method of the invention. To this end, the circular vector is cut with two restriction enzymes, for example NheI and BamHI, which produce staggered ends which are not complementary to one another. The staggered ends are partially filled in by treatment with Klenow enzymes. The sequence of the oligonucleotides or amplification products to be cloned into the vector is defined in such a way that they have, as double-stranded fragment, protruding 5′ ends complementary to the vector and can be ligated with said vector.

### EXAMPLE 1

### Mutagenesis

1.1 Mutagenesis PCR

The conditions for the PCR were chosen according to the following table. Since amplification of an entire plasmid results in long PCR products, the reaction mixtures were supplemented by 5% by volume of glycerol and 2-5% by volume of DMSO. Moreover, both the amount of template DNA (<10 ng) and the number of PCR cycles (≦25) were kept as small as possible.

Mutagenesis PCR mixture

| Substance | Concentration/amount |
|---|---|
| Template DNA | <10 ng |
| Buffer | 1x |
| dNTPs | 0.3 μM each |
| Oligonucleotides | 0.3-0.6 μM each |
| Glycerol | 5% (Karasavvas and Zakeri, 1999) |
| DMSO | 2-5% (Karasavvas and Zakeri, 1999) |
| MgCl₂ | 2.25 mM |
| Expand ™ | 1.75 U |
| H₂O | to 50-100 μl |

Cyclic amplification:

| Temperature | Duration | Number of cycles |
|---|---|---|
| 94° C. | 2 min | 1 |
| 94° C. | 30 sec | |
| 50-68° C. | 1 min | ≦25 |
| 68° C. | 1 min/kb | 1 |
| 68° C. | 10 min | |

Other alternatives are variations in MgCl₂ concentration or/and the use of HotStart polymerases. It should be noted that generation and use of very pure PCR products were preconditions for successful mutagenesis. Subsequent purification and T₄ reaction were carried out according to Example 2.2.2.

1.2 Ligation and DpnI Digestion

Between 100 and 200 ng of DNA and 0.5 μl of ligase (Invitrogen) were used for ligation in a total volume of 10 μl. The negative control used was a ligation mixture without ligase. After incubation at 4° C. overnight, the entire ligation mixture was admixed with 2 μl of buffer A (Roche), 0.5 μl of DpnI and 7.5 μl of H₂O, and the template DNA was digested at 37° C. for 30 min. Transformation into E. coli was carried out under standard conditions. Provided that the number of transformants of the mixture with ligase was more than 5 times the number of transformants of the mixture without ligase, DNA was isolated from 2 clones and the mutagenesis was verified by sequencing.

### EXAMPLE 2

### Cloning of Oligonucleotides

2.1 Preparation of the Vector According to Variant 1: Opening of the Vector by Restriction Digestion with Subsequent Klenow Reaction

2.1.1 Restriction Digestion

The cloning vectors were opened at the appropriate sites with the aid of restriction enzymes. Since not all enzymes cut in the same buffer and since some enzymes work only very poorly close to DNA ends, the conditions for the restriction digestion were set according to the choice of enzymes. The conditions for cutting with HindIII and BamHI are indicated below. First, approx. 7 μg of the vector were digested with 10 U of HindIII in 30 μl of 1× buffer A (Roche) in an incubator at 37° C. for one hour. The addition of another 5 U of HindIII was followed by cutting for another hour.

The buffer was changed either by adding the appropriate buffer and salt solutions or, as in this case, by ethanol precipitation (see 2.1.3) and subsequent resuspending in the buffer for the second enzyme. In the example described here, the vector was digested in 1× buffer B (Roche) and 10 U of BamHI in a total volume of 30 μl for one hour and, after another addition of enzyme (5 U), overnight. On the next morning, 5 U of BamHI were added, followed by another incubation for one hour. The restriction enzymes were then denatured at 65° C. for 10 min.

2.1.2 Klenow Reaction

The following solutions were pipetted to the restriction mixture:

| | | Final |
|---|---|---|
| Reagent | Volume | concentration |
| Appropriate dNTPs | 4 μl | 1 mM each |
| (10 mM each, here dATP, dGTP) | | |
| Klenow enzyme (2 U/μl) | 2 μl | 4 U/reaction |
| H₂O | to 40 μl | |

It had to be taken into account here that the total enzyme volume did not exceed 10% of the amount of solution, because the high glycerol content could have reduced the enzyme activity. The mixture was incubated at room temperature for 30 min. and the Klenow enzyme was then denatured at 75° C. for 15 min.

2.1.3 Purification

In order to remove the enzymes from a reaction mixture, two phenol/chloroform extractions were carried out. To this end, 10 μl of 3 M sodium acetate pH 5.2, 50 μl of H₂O and 100 μl of phenol/chloroform were added to the DNA solution. After vigorous shaking and centrifugation in a bench centrifuge at 13 000 rpm for three minutes, the upper, aqueous phase was removed and again purified with phenol/chloroform. The DNA was precipitated by adding 250 μl of ethanol (analytical grade) and incubated at −70° C. for 15 min. The DNA was then pelleted by centrifugation at 13 000 rpm and 4° C. for 15 min., washed with 70% ethanol and dried at 37° C. The vector prepared in this way was resuspended in 30 μl of H₂O and the concentration was estimated by way of agarose gel electrophoresis.

2.2 Preparation of the Vector According to Variant 2: Generation of the Opened Vector by PCR with Subsequent T₄ DNA-Polymerase Reaction Using Particular Stop Nucleotides

2.2.1 Polymerase Chain Reaction (PCR)

For amplification, the PCR mixture below was pipetted and the PCR was carried out according to the program indicated. Since amplification of virtually the entire plasmid results in long PCR products, the reaction mixtures were supplemented with 5% by volume of glycerol and 2-5% by volume of DMSO. Moreover, both the amount of template DNA (<10 ng) and the number of PCR cycles (≦25) were kept as small as possible.

Mutagenesis PCR mixture

| Substance | Concentration/amount |
|---|---|
| Template DNA | <10 ng |
| Buffer | 1x |
| dNTPs | 0.3 μM each |
| Oligonucleotides | 0.3-0.6 μM each |
| Glycerol | 5% (Karasavvas and Zakeri, 1999) |
| DMSO | 2-5% (Karasavvas and Zakeri, 1999) |
| MgCl₂ | 2.25 mM |
| Expand ™ | 1.75 U |
| H₂O | to 50-100 μl |

Cyclic amplification:

| Temperature | Duration | Number of cycles |
|---|---|---|
| 94° C. | 2 min | 1 |
| 94° C. | 30 sec | |
| 50-68° C. | 1 min | ≦25 |
| 68° C. | 1 min/kb | 1 |
| 68° C. | 10 min | |

2.2.2 Purification

The PCR products were purified using the Qiagen PCR purification kit. For this purpose, the PCR products were mixed with 5 volumes of buffer PB and bound to a minicolumn by way of centrifugation. After washing with PE buffer, the DNA was eluted with 40 μl of H₂O.

2.2.3 Trimming of the PCR Product

The protruding 5′ ends complementary to the prepared vector were generated by preparing the following mix on ice:

| | | Final | |
|---|---|---|---|
| Substance | Volume (μl) | concentration | |
| DNA | 20 | 2.5-5 | μg/40 μl |
| T₄ incubation buffer | 8 | 1x | |
| dNTP (10 mM) | 4 | 1 | mM each |
| Water | to 38 μl | | |
| The mixture was mixed well before adding T₄ DNA polymerase | | | |
| T₄ DNA polymerase (1 U/μl) | 2 | 0.05 | U/μl |

After incubation at 12° C. for 30 minutes, the samples were denatured at 75° C. for 15 min. and purified and analyzed, as described for preparation of the vector.

2.3 Introduction of Double-Stranded Oligo-Nucleotides

2.3.1 Preparing the Double-Stranded Oligonucleotides

For oligonucleotide dimerization, 10 μg of each primer in a 50 mM Tris-HCl buffer (pH 7.5) were mixed with 1.75 mM MgCl₂ in a final volume of 100 μl and denatured at 95° C. in a PCR apparatus. The hybridization was carried out by slowly cooling the heating block to room temperature.

2.3.2 Ligation

The prepared vector and double-stranded oligo-nucleotides were mixed in a final volume of 10-15 μl so that the ratio of free ends was 1:500, using approx. 100 ng of vector. After addition of 0.5 μl of T₄ DNA ligase, the mixture was incubated at 4° C. overnight.

### EXAMPLE 3

### XcmI Cloning

3.1 Introduction

The use of restriction enzymes whose cleavage site is not located in the recognition sequence for opening the cloning vector has several far-reaching advantages. The most important advantage is the possibility of directed cloning using only one restriction enzyme. Since one restriction is substantially more efficient than a double digestion, the rate of cloning increases enormously. Moreover, a linker fragment is not deleted from the vector and therefore need not be removed. In the case of the XcmI recognition sequence used here, nine bases at the cleavage site can be freely chosen. However, it would also be possible to use any other restriction enzyme which, apart from a specific recognition sequence, permits the free choice of bases. Examples are XmnI, Van91I, SfiI, MwoI and others. This enables the user to introduce bases which allow utilization of oligonucleotides already present in the laboratory. The high efficiency achieved by this cloning technique renders the latter suitable for automated cloning of DNA.

3.2 Generation of pDLO12-XcmI

The XcmI cleavage site was introduced into the pDLO12 vector by way of a Di-/TriSec mutagenesis using the oligonucleotides pDLO1x-5′-XcmI (5′-phos-TTATCGATGGATCCAGACATGATAAGATACATTGA-3′) and pDLO1-3′-XcmI (5′-phos-ATAAGGTGGCAGGTCGGATCGGTCC-3′). The pDLO12 vector was amplified with the aid of these oligonucleotides. After T₄ DNA-polymerase reaction with dCTP and dGTP, ligation and subsequent transformation into E. coli, the positive clones were identified by way of XcmI digestion. Introduction of the cleavage site was moreover checked by means of sequencing.

Cloning of G3BP into pDLO-XcmI:

XcmI has the following recognition sequence:

```
XcmI
                    ↓
5′-CCANNNNNNNNNTGG-3′
3′-GGTNNNNNNNNNACC-5′
                  ↑
```

The bases indicated by N are freely choosable and do not contribute to recognition of the cleavage sites. This makes it possible to introduce a sequence which is required for a Di-TriSec cloning by way of a particular, often used cloning strategy. This has the advantage that only one primer pair needs to be purchased which then, however, allows cloning into various vectors. In the example illustrated here, the XcmI cleavage site has the following sequence:

```
XcmI
                    ↓
5′-CCAccttatcgaTGG-3′
3′-GGTggaatagctACC-5′
                  ↑
```

XcmI digestion of the vector produces a “single-base protruding 3′ end”.

| | |
|---|---|
| 5′-CCAcctta | tcgaTGG-3′ |
| 3′-GGTggaa | tagctACC-5′ |

Subsequently, the trimming reaction (3′-5′-exonuclease activity) is carried out using T4 DNA polymerase and dCTP as stop nucleotide:

| | |
|---|---|
| 5′-CCAcc | tcgaTGG-3′ |
| 3′-GGTggaa | ctACC-5′ |

It is then possible to clone PCR fragments into the purified vectors, which fragments have the sequence “TTC” at the 5′ end and “GAC” at the 3′ end (in the example: G3BP cloning using the oligonucleotides 5′-G3BP-DLO (5′-TTC ATG GTG ATG GAG AAG CCT AGT-3′) and 3′-G3BP (5′-GAC TTA CTG CCG TGG CGC AAG CCC CCT-3′). The PCR product carries the following ends:

5′-TTCxxxxxxxxxxxxxxxxGTC-3′
3′-AAGxxxxxxxxxxxxxxxxCAG-5′

It is likewise subjected to a T4 DNA-polymerase reaction using dGTP as stop nucleotide, thereby producing the product below:

5′-TTCxxxxxxxxxxxxxxxxG-3′
3′-GxxxxxxxxxxxxxxxxCAG-5′

After purification, the vector and the insert may be ligated.

5′-CCAccTTCxxxxxxxxxxxxxxxxGtcgaTGG-3′
3′-GGTggaaGxxxxxxxxxxxxxxxxCAGctACC-5′

The ligated DNA is transformed into E. coli. The positive clones may be identified and checked with the aid of colony PCR and sequencing.

3.3 Methods:

3.3.1 Amplification of the Insert:

Template: G3BP in pET28a (GI: 5031702)

PCR: 3 × mixtures of 50 μl each

| | | |
|---|---|---|
| 1 | μl | of pARB021 (10 ng/μl) |
| 15 | μl | of Expand buffer 3 (Roche) |
| 3 | μl | of dNTPs (10 mM each) |
| 1.5 | μl | of 5′ primer (50 μM) |
| 1.5 | μl | of 3′ primer (50 μM) |
| 1 | μl | of Expand long template |
| 125 | μl | of H₂O |
| 150 | μl | |

$$Program \text{:} \\ {1}^{′} {94}^{∘} C . 1 \times \\ \begin{matrix}\begin{matrix}{1}^{′} {94}^{∘} C . \\ {1}^{′} {55}^{∘} C .\end{matrix} \\ {2}^{′} {68}^{∘} C .\end{matrix} \} 25 \times$$

3.3.2 Vector Digestion with XcmI

10 μg of pDLO12-XcmI

2 μl of XcmI

5 μl of Stratagene “one-for-all” buffer to 50 μl

approx. 2 h, 37° C.

3.3.3 Vector Trimming with T4 DNA Polymerase:

2.5 μl of cut pDLO12-XcmI (0.5 μg/μl)

2 μl of buffer A (Roche)

2 μl of BSA (1:10)

2 μl of dCTP (10 mM)

10.5 μl of H₂O

1 μl of T4 (1:5)

30 min at 12° C., then 15 min at 75° C.

3.3.4 Insert Trimming with T4 DNA Polymerase:

8 μl of G3BP PCR fragment (150 ng/μl)

4 μl of buffer A (Roche)

0.4 μl of BSA (100×)

4 μl of dGTP (10 mM)

22.6 μl of H₂O

1 μl of T4 polymerase

30 min at 12° C., then 15 min at 75° C.

Trimming was followed by a purification via phenol/chloroform extraction and ethanol precipitation (see section 2.1.3).

3.3.5 Ligation (in 15 μl)

3.5 μl of trimmed pDLO12-XcmI vector (100 ng)

1 μl of trimmed G3BP PCR fragment (100 ng)

3 μl of 5× ligation buffer

0.5 μl of T4 ligase (Life Tech.)

8 μl of H₂O

4° C. overnight

The DNA was then transformed into E. coli. The positive clones were identified by colony PCR and restriction digestion and verified by sequencing.

### EXAMPLE 4

### Di-TriSec Cloning of DNA Oligonucleotides into a Vector for in Vivo Expression of siRNA

4.1 Introduction

The Di-/TriSec cloning developed is to be studied for its suitability for cloning of DNA oligonucleotides into vectors which are suitable for producing double-stranded RNA in transfected cells. An inhibitor of the lamin A/C gene was to be generated by way of example.

4.2 Methods

The DNA oligos having the sequence hLamin 1as IVE 5′ CTT TTC CAA AAA CTG GAC TTC CAG AAG AAC ATC TCT TGA ATG TTC TTC TGG AAG TCC AGG GG 3′ and hLamin 1s IVE 5′ TCC CCC TGG ACT TCC AGA AGA ACA TTC AAG AGA TGT TCT TCT GGA AGT CCA GTT TTT GGA AA 3′ were dissolved at a concentration of 100 μM in H₂O.

For dimerization, complementary oligonucleotides were mixed at different concentrations in a magnesium-containing buffer (see section 2.3.1) and heated to 95° C. in a PCR apparatus. The oligonucleotides were hybridized by slow cooling from 95° C. to room temperature over 3 hours. The lid of the reaction vessel remained heated to 95° C. The oligonucleotides may be dimerized at concentrations of 0.25-40 μM, this having no decisive influence on the rate of ligation.

The cloning was prepared by cutting the pSUPER vector (Brummelkamp et al., 2002) with the restriction endonucleases BgIII and HindIII according to the manufacturer's instructions and filled in with dATP and dGTP according to the above-described method (see section 2.1.1).

The ligation was carried out as described above (see section 2.3.2), assaying ratios of free vectors ends to free insert ends of 1:100 and 1:500.

The ligation inserts were transformed into E. coli by means of standard methods, the recombinant plasmids were propagated and purified.

4.3 Result

The efficiency of cloning the double-stranded hLamin 1 IVE oligonucleotide was 84% overall, the ratio of vector to insert having only little influence (1:500, 80%, 1:100, 86%).

The example depicted here emphasizes the high efficiency of the cloning method of the invention. The small influence of the concentration of the oligonucleotides on dimerization and of the ratio vector:insert on the rate of ligation confirms the robustness of the method with respect to the reaction conditions.

In the postgenomic era, there is a steadily increasing need for approaches in which a very large number of genes is studied. The siRNA cloning technique depicted here could be an important step for generating a permanent “knockdown” cell clone library. All steps, starting with dissolving the oligonucleotides via dimerization, ligation, transformation to PCR screening for positive clones and isolation of plasmid DNA may be automated, thereby generating silencer constructs for virtually all human genes over a short period.

The advantage of the method described here compared to conventional methods is high efficiency and robustness of the cloning. Conventional strategies are based on cloning double-stranded oligonucleotides via palindromic restriction cleavage sites, with the oligonucleotides to be cloned forming preferably concatamers. The result is a considerably reduced cloning efficiency. In contrast, the method described herein rules out concatamer formation of the oligonucleotides and thereby increases the efficiency of cloning.

### References

Cooney, A. J. (1998). Use of T4 DNA polymerase to create cohesive termini in PCR products for subcloning and site-directed mutagenesis. Biotechniques, 24, 30, 32, 34

Deng, W. P. and Nickoloff, J. A. (1992). Site-directed mutagenesis of virtually any plasmid by eliminating a unique site. Anal. Biochem., 200, 81-88

Hutchison, C. A., Phillips, S., Edgell, M. H., Gillam, S, Jahnke, P. and Smith, M. (1978). Mutagenesis at a specific position in a DNA sequence. J. Biol. Chem., 253, 6551-6560

Karasavvas, N. and Zakeri, Z. (1999). Relationships of apoptotic signaling mediated by ceramide and TNF-alpha in U937 cells. Cell Death and Differentiation, 6, 115-123

Kleina, L. G., Masson, J. M., Normanly, J., Abelson, J. and Miller, J. H. (1990). Construction of Escherichia coli amber suppressor tRNA genes. II. Synthesis of additional tRNA genes and improvement of suppressor efficiency. J Mol Biol, 213, 705-717

Kramer, B., Kramer, W. and Fritz, H. J. (1984). Different base/base mismatches are corrected with different efficiencies by the methyl-directed DNA mismatch-repair system of E. coli. Cell, 38, 879-887

Lottspeich, F. and Zorbas, (1998). H. Bioanalytik. Spektrum Akademischer Verlag. Ref type: book, whole

Normanly, J., Kleina, L. G., Masson, J. M., Abelson, J. and Miller, J. H. (1990). Construction of Escherichia coli amber suppressor tRNA genes. III. Determination of tRNA specificity. J Mol Biol, 213, 719-726

Zhu, L. (1996). In vitro site-directed mutagenesis using the unique restriction site elimination (USE) method. Methods Mol Biol, 57, 13-29

1-18. Canceled

19. A method for cloning nucleic acid fragments into a vector, which comprises the following steps:
(a) providing a circular double-stranded nucleic acid vector,
(b) cleaving the vector of (a) with two different restriction endonucleases, obtaining a linear vector having two different staggered ends,
(c) partially filling in the ends of the cut vector, generating a filled-in linear vector having different staggered ends which are neither complementary to one another nor self-complementary,
(d) providing a nucleic acid fragment to be cloned into the vector of (c), which has two different staggered ends which are complementary to the ends of the filled-in vector but which are neither complementary to one another nor self-complementary, and
(e) ligating said nucleic acid fragment into said filled-in vector.

20. The method as claimed in claim 19,
characterized in that
step (b) uses restriction enzymes which produce protruding 5′ ends.

21. The method as claimed in claim 19,
characterized in that
the filling-in in step (c) comprises a treatment with an enzyme having DNA-polymerase activity, in particular with Klenow enzyme, in the presence of no more than two nucleotide triphosphates, in order to effect only partial filling-in of the staggered ends.

22. The method as claimed in any of claims 19,
characterized in that
the nucleic acid fragment to be cloned to the vector is generated by chemical or enzymic synthesis of single-stranded oligonucleotides and subsequent hybridization of said single-stranded oligonucleotides.

23. A reagent kit, in particular for carrying out the cloning methods as claimed in claim 19, which kit comprises:
(a) means for cleaving a nucleic acid by two different restriction endonucleases which generate in each case different staggered ends,
(b) means for partial filling-in of staggered ends in double-stranded nucleic acids for generating staggered ends which are neither complementary to one another nor self-complementary,
(c) means for hybridizing DNA oligonucleotides and
(d) means for ligating nucleic acids.

24. A method for cloning nucleic acid fragments into a vector, which comprises the following steps:
(a) providing a circular double-stranded nucleic acid vector,
(b) providing two oligonucleotides which
(i) in each case hybridize with one strand of the nucleic acid vector and
(ii) have in the region of their 5′ ends a section of 2-4 base pairs complementary to one another,
(c) amplifying the nucleic acid vector of (a) by using the oligonucleotides of (b) as primers, obtaining a double-stranded amplification product with staggered ends,
(d) trimming the ends of said amplification product, generating a trimmed linear vector having different staggered ends which are neither complementary to one another nor self-complementary,
(e) providing a nucleic acid fragment to be cloned into said vector, which has two different staggered ends which are complementary to the ends of the trimmed vector but are neither complementary to one another nor self-complementary, and
(f) ligating said nucleic acid fragment into the said trimmed vector.

25. The method as claimed in claim 24,
characterized in that
amplification in step (c) comprises a PCR.

26. The method as claimed in claim 24,
characterized in that
trimming in step (d) comprises a treatment with an enzyme having 3′ exonuclease activity, in particular with T₄ DNA polymerase in the presence of stop nucleotides.

27. A reagent kit, in particular for carrying out the cloning method as claimed in claim 24, which kit comprises:
(a) means for nucleic acid amplification,
(b) means for trimming double-stranded nucleic acids to generate staggered ends which are neither complementary to one another nor self-complementary,
(c) means for hybridizing DNA oligonucleotides and
(d) means for ligating nucleic acids.

28. A method for cloning nucleic acid fragments into a vector, which comprises the following steps:
(a) providing a circular double-stranded nucleic acid vector having a restriction cleavage site for a restriction enzyme, at which site the recognition sequence is located beside the actual cleavage site,
(b) cleaving the vector with said restriction enzyme,
(c) partially filling in or trimming the ends of the cut vector, generating a linear vector having different staggered ends which are neither complementary to one another nor self-complementary,
(d) providing a nucleic acid fragment to be cloned into the vector of (c), which has two different staggered ends which are complementary to the ends of the filled-in vector but which are neither complementary to one another nor self-complementary, and
(e) ligating said nucleic acid fragment into said filled-in vector.

29. A reagent kit, in particular for carrying out the cloning method as claimed in claim 28, which kit comprises:
(a) means for cleaving a nucleic acid by a restriction endonuclease having a restriction cleavage site at which the recognition sequence is located beside the actual cleavage site,
(b) means for partial filling-in of staggered ends in double-stranded nucleic acids for generating staggered ends which are neither complementary to one another nor self-complementary,
(c) means for trimming double-stranded nucleic acids to generate staggered ends which are neither complementary to one another nor self-complementary,
(d) means for hybridizing DNA oligonucleotides and
(e) means for ligating nucleic acids.

30. A method for mutagenizing nucleic acids, which comprises the following steps:
(a) providing a circular double-stranded nucleic acid template to be mutagenized,
(b) providing two oligonucleotides which
(i) hybridize in each case with one strand of said nucleic acid template,
(ii) have in the region of their 5′ ends a section of 2-4 base pairs complementary to one another, and
(iii) with at least one of said oligonucleotides comprising the mutated target sequence,
(c) amplifying the nucleic acid template to be mutagenized of (a) by using the oligonucleotides having the mutated target sequence of (b) as primers, obtaining a mutated double-stranded amplification product,
(d) trimming the ends of said amplification product, generating a trimmed amplification product having protruding 5′ ends complementary to one another, and
(e) ligating said trimmed amplification product, obtaining a mutated circular nucleic acid construct.

31. The method as claimed in claim 30,
characterized in that
amplification in step (c) comprises a PCR.

32. The method as claimed in claim 30,
characterized in that
trimming in step (d) comprises a treatment with an enzyme having 3′ exonuclease and polymerase activity, in particular with T₄ DNA polymerase in the presence of stop nucleotides.

33. The method as claimed in claim 30,
characterized in that
the nucleic acid template is removed prior to ligation in step (e).

34. The method as claimed in claim 33,
characterized in that
a methylated nucleic acid template and, for its removal, a restriction endonuclease cleaving only methylated DNA are used.

35. The method as claimed in claim 30,
characterized in that
the mutagenization is selected from:
(i) effecting one or more single nucleotide substitutions,
(ii) effecting one or more single nucleotide insertions,
(iii) effecting one or more inversions of a sequence of at least two bases,
(iv) effecting one or more single nucleotide deletions, and
(v) effecting any combination of mutagenizations (i), (ii), (iii) and (iv).

36. A reagent kit, in particular for carrying out the mutagenization method as claimed in claim 30, which kit comprises:
(a) means for nucleic acid amplification,
(b) means for trimming double-stranded nucleic acids to generate staggered ends complementary to one another,
(c) means for ligating nucleic acids and,
(d) where appropriate, means for selectively removing methylated DNA.
